(19) United States
(12) Patent Application Publication
(10) Pub. No.: US 2015/0122261 A1
(43) Pub. Date: May 7, 2015

(54) RESPIRATORY THERAPY APPARATUS

(71) Applicant: SMITHS MEDICAL INTERNATIONAL LIMITED, Kent (GB)

(72) Inventor: Emily Pettitt, Bristol (GB)

(73) Assignee: SMITHS MEDICAL INTERNATIONAL LIMITED, Kent (GB)

(21) Appl. No.: 14/401,879

(22) PCT Filed: May 14, 2013
(86) PCT No.: PCT/GB2013/000214

§ 371 Date: Nov. 18, 2014

(30) Foreign Application Priority Data
Jun. 6, 2012 (GB) 1209962.8

(51) Int. Cl. A61M 16/04; A61M 16/08; A61M 16/20; A61M 16/00

(52) U.S. Cl. CPC A61M 16/049; A61M 16/0003; A61M 16/0063; A61M 16/0816; A61M 16/204; A61M 2205/3592; A61M 2205/583; A61M 2205/587; A61M 2205/52; A61M 2230/42
USPC 128/205.24; 128/205.25

(57) ABSTRACT

An inspiratory exerciser apparatus has a loop shape housing with a mouthpiece 1, an air inlet 2, an outlet 3 and a spring-loaded valve 4 that provides a restriction to inhalation through the inlet. The apparatus includes an electronics unit 60 that records use of the apparatus for download to a remote computer, such as via a connector port 65 or by wireless means. The electronics unit 60 also causes lamps 63 in a translucent part of the housing 11 to flash during the prescribed exercise period to encourage the patient to inhale and exhale at a prescribed rate and for a prescribed time.

This invention relates to respiratory therapy apparatus of the kind including a mouthpiece, an air opening and a restriction to flow of air between the mouthpiece and the opening against which the patient breathes.

The apparatus is more particularly concerned with apparatus for use in improving the performance of inspiratory muscles such as in patients suffering from COPD or similar respiratory problems, or for athletes and others seeking to reduce breathlessness.

Apparatus is presently available for training inspiratory muscles, incorporating some form of resistance against which the user inhales. By using such apparatus repeatedly over a prolonged period it is possible to improve lung function. Apparatus presently available includes the Powerbreathe and Powerbreathe Kinetic from Powerbreathe Medic, PowerLung from PowerLung Inc, Ultrabreathe from Tangent Healthcare Ltd, and' Voldyne 5000 from Hudson RCI. Respiratory therapy apparatus is also described in U.S. Pat. No. 6,553,746, GB2278545A, GB2451593B, U.S. Pat. No. 6,726,598, U.S. Pat. No. 4,854,574 and U.S. Pat. No. 4,533,137.

Presently available apparatus suffers from various problems such as being difficult to use by patients with weak hands. Another major problem is that users often fail to use conventional apparatus regularly for the correct period and so achieve little or no benefit.

It is an object of the present invention to provide alternative respiratory apparatus.

According to one aspect of the present invention there is provided respiratory therapy apparatus of the above-specified kind, characterised in that the apparatus includes means to record use of the apparatus and provide an indication thereof.

The apparatus preferably includes means by which the record of use of the apparatus can be downloaded to a remote computer. The means by which the record of use can be downloaded is preferably selected from one or more of the following: an electrical connector port, an infra-red transmitter and a radio transmitter. The air opening is preferably an inlet such that the patient can inhale air from the opening via the restriction. The restriction may include a valve with a spring urging a valve member against a valve seat. The force with which the valve member is urged against the valve seat may be adjustable by rotating a part of the valve. The apparatus is preferably arranged to indicate the duration of the prescribed therapy period, such as by visual means that changes between an alternating and a steady state. The visual means may be arranged to alternate during the prescribed therapy period and to change to a steady state when the prescribed therapy period has ended. The visual means preferably includes a lamp located within a translucent part of the housing of the apparatus so that the translucent part is illuminated by the lamp. The steady state is preferably an illuminated state of a predetermined duration. The apparatus preferably includes a display for representing a count of the number of breaths taken during the therapy period, the display preferably being positioned such that it is not visible by the patient during the therapy period.

The therapy apparatus is preferably of a generally looped shape including a first arm containing the air opening and the restriction to flow of air, a second arm including a second air opening and the means to record use of the apparatus, the second arm being attached at one end with the first arm, extending alongside the first arm and being spaced from it to provide an open loop by which the apparatus can be supported, and the mouthpiece extending generally in a direction opposite from the loop. The first and second arms are preferably arranged such that the apparatus can be supported with both arms resting on a support surface and with the mouthpiece being spaced above the surface.

Respiratory apparatus according to the present invention will now be described, by way of example, with reference to the accompanying drawings, in which:

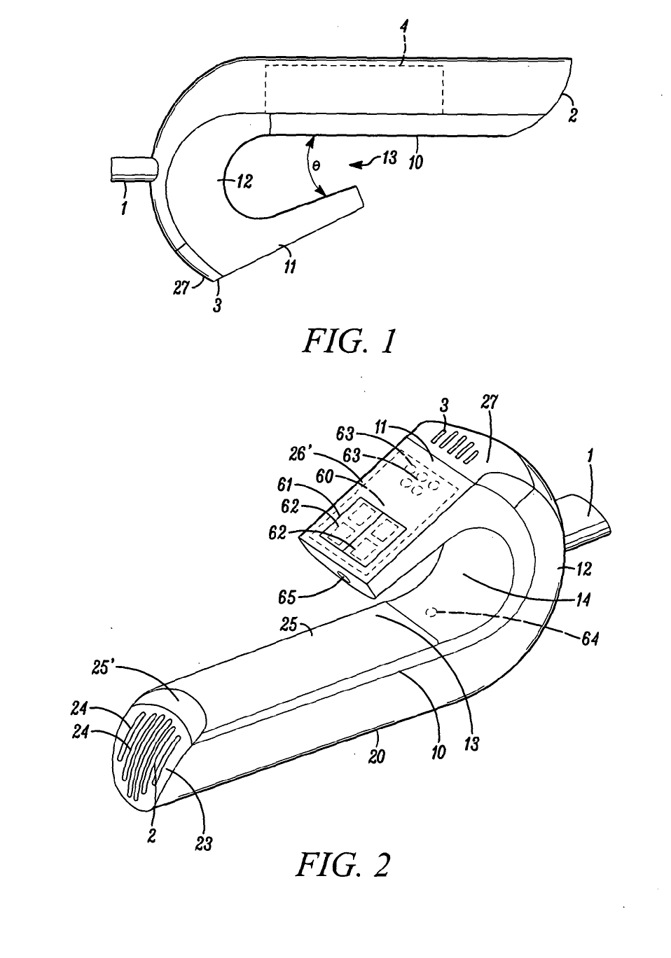

FIG. 1 is a side elevation view of the apparatus;

FIG. 2 is a perspective view of the underside of the apparatus;

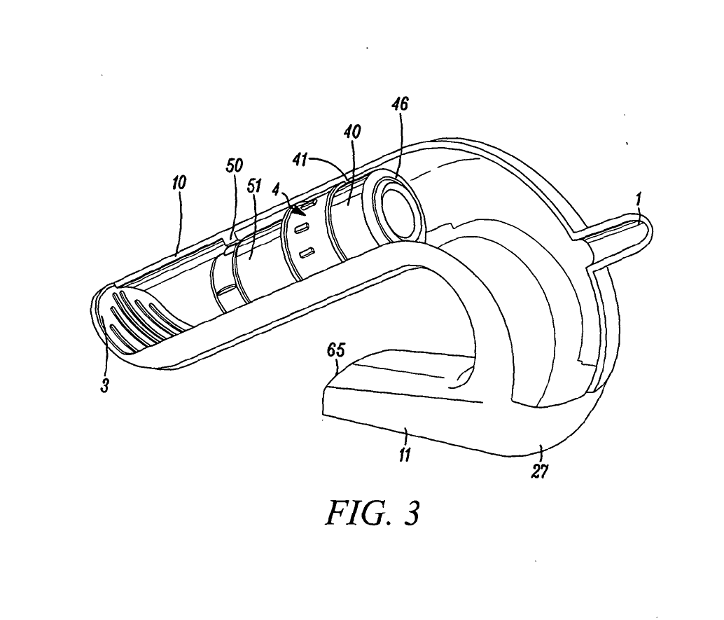

FIG. 3 is a perspective view of the apparatus resting on a flat surface;

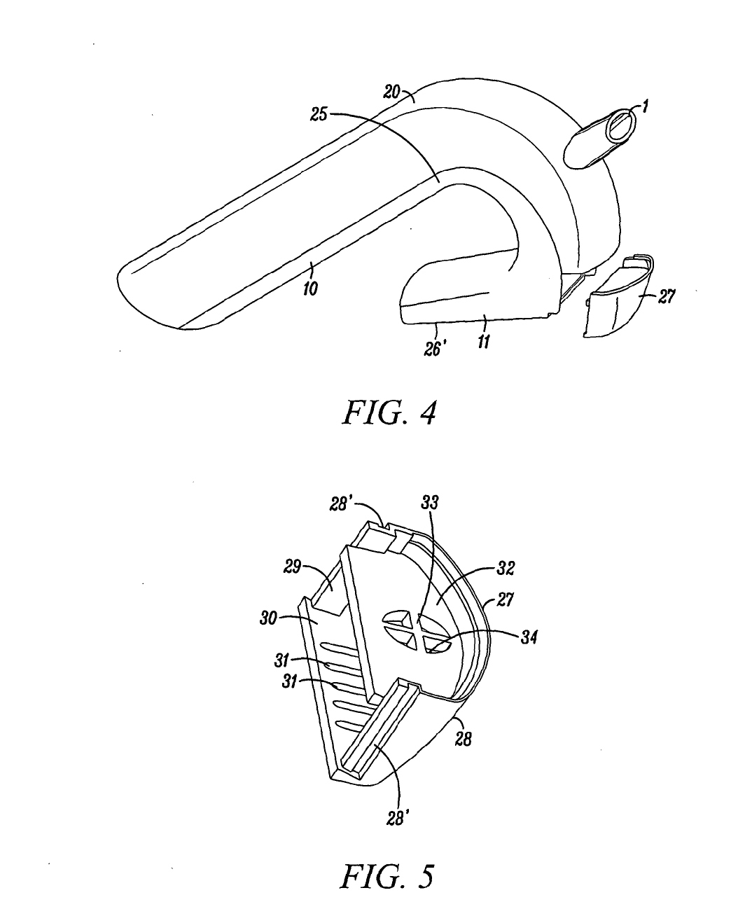

FIG. 4 is a perspective view of the apparatus showing the saliva trap removed from the apparatus;

FIG. 5 is a perspective view of the saliva trap;

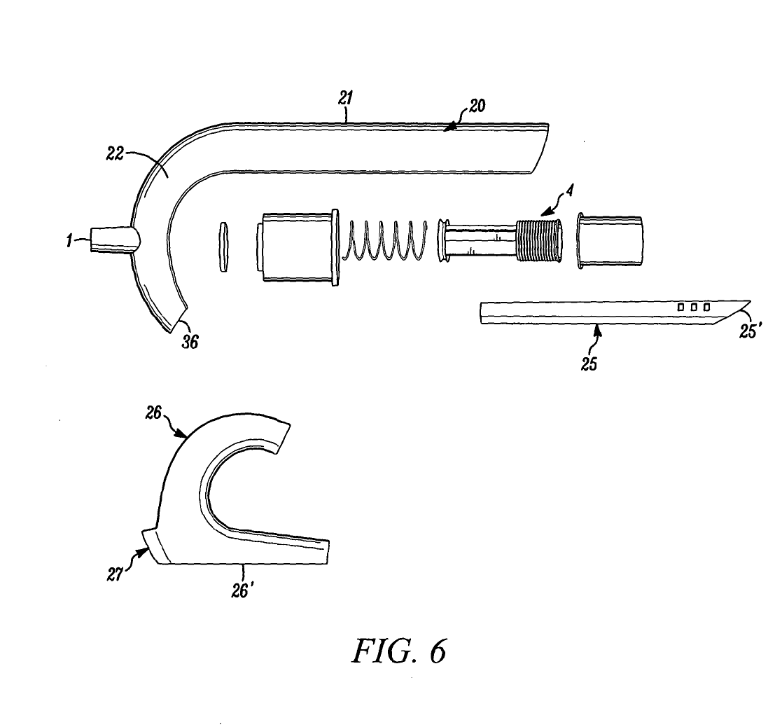

FIG. 6 is an exploded view of the apparatus showing most of its components; and

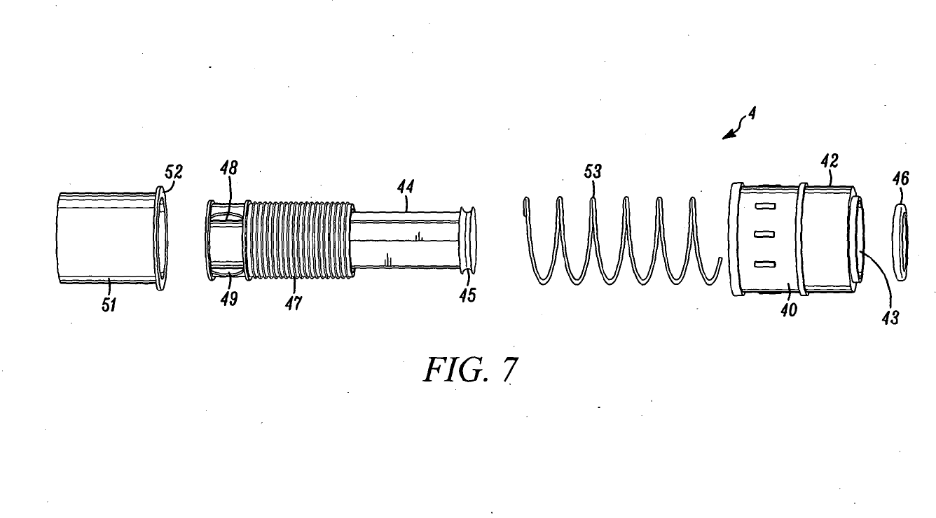

FIG. 7 is an exploded view of an inhalation valve used in the apparatus.

With reference first to FIGS. 1, 2 and 3, the apparatus has a mouthpiece 1, an air inlet 2, an air outlet 3 and a one-way inspiratory valve assembly 4 between the inlet and the mouthpiece. The valve assembly 4 provides a resistance to flow so that the user has to exert force to inhale air via the mouthpiece 1 before expelling the air through the air outlet 3. In this way, the user can exercise his inspiratory lung muscles.

It can be seen that the apparatus has a generally looped or P shape with the air inlet 2 being located in an upper, inlet arm or handle portion 10 and the air outlet 3 being located in a lower, electronics housing 11. The lower housing 11 is connected with the handle portion 10 by a curved loop portion 12 so that the lower housing extends along and towards the handle portion at an angle θ of about 20°. The handle portion 10 is typically about 35 mm wide, the lower housing 11 terminating about half way along the length of the upper portion and being spaced from it by a gap 13, about 30 mm wide, to form an open loop 14.

With reference now also to FIG. 6, the apparatus is made of several plastics mouldings, such as of polycarbonate, clipped together in a gas-tight manner. The handle portion 10 is provided by a first transparent moulding 20 having a straight portion 21 with a curved section 22 extending around about 270°. The curved section 22 includes the mouthpiece 1 integrally formed with it and projecting rearwardly. The mouthpiece 1 tapers slightly at an angle of 4.3° and its width is about two thirds the width of the apparatus. This ensures that, if the apparatus is laid on a surface on its side, the mouthpiece 1 does not contact the surface, thereby preventing contamination. As can be seen in FIG. 2, the forward end 23 of the first moulding 20 is closed apart from several parallel slots 24, which provide the air inlet 2 of the apparatus. The handle portion 10 is completed by a second moulding 25, of an opaque polycarbonate, which is straight and substantially flat. The second moulding 25 is clipped to the underside of the straight portion 21 of the first moulding 20 and can be slid forwardly along the first moulding to enable access to the inspiratory valve assembly 4.

A third moulding 26 has a generally U shape providing the lower, electronics housing 11 and containing the electronics and display (to be described later) of the apparatus. The moulding 26 provides a flat, lower face 26′ for the housing 11. The third moulding 26 clips to the inside of the curved rear end 22 of the first moulding 20 and abuts the rear end of the second moulding 25. The casing of the apparatus is completed by a fourth moulding 27 (FIGS. 4 and 5) in the form of a box-like structure forming a saliva trap of the apparatus. The saliva trap 27 has a curved front face 28 forming a continuation of the curved, outer surface of the first moulding 20 and an open rear face 29. The sides of the front face 28 have ribs 28′ that enable the trap 27 to be slid down into place in the electronics housing 11. The trap 27 also has a flat lower face 30 forming a continuation of the lower face 26′ of the housing 11, this lower face having a series of slots 31 formed in it to provide the air exit outlet 3. The trap 27 further includes an upper plate 32 with a central opening 33 supporting a one-way flap valve 34, which permits air to flow into the trap and out through the slots 31 but prevents air being drawn into the apparatus via the trap when the user inhales. The opening 33 aligns with an open rear end 36 of the first moulding 20.

The shape of the apparatus has been chosen to make it easier to use. The flat lower face 26′ of the electronics housing 11 is inclined parallel with an angled face 25′ at the forward end of the second moulding 25 to enable the apparatus to be stood stably in an upright position, as shown in FIG. 3, resting on these two surfaces. The slanted handle 10 forms a gap under the apparatus that makes it easy to pick up intuitively. The angled orientation of the mouthpiece 1 directs it away from surfaces to avoid contaminating either the product or the surrounding environment. The forward end 23 of the first moulding 20 through which the inlet slots 24 open is inclined away from the supporting face 25′ so that the slots do not make contact with the support surface. The looped shape of the apparatus enables the user to clutch it or rest it in his hand if he becomes tired or has poor dexterity, such as caused by arthritis or other ailments. The shape enables the apparatus to be held loosely in the fingers without being dropped. The hooked shape also allows the apparatus to be hooked over a rail, equipment, furniture or the like to allow the user to take a break from holding the apparatus and yet still have it readily available

With reference now especially to FIG. 7, the inspiratory valve assembly 4 is contained within the handle portion 10 trapped between the first and second mouldings 20 and 21. The valve assembly 4 includes a cylindrical valve housing 40 fixed longitudinally in position in the handle portion 10 by surface formations 41 on its inside surface. These formations 41, however, allow the valve housing 40 to be rotated. At its rear, right-hand end 42, the housing 40 has a rearwardly-facing valve seat 43. The valve assembly 4 also includes a valve member or compression column 44 extending coaxially within the housing 40 and movable along its length relative to the housing. The rear end of the column 44 is moulded with an annular groove 45 embraced by an O-ring seal 46. When the column 44 is in a forward position the seal 46 engages the seat 43 in the housing 40 to prevent air flow between the two components, and hence to prevent any air flow along the handle portion 10. The column 44 is also formed with an external screw thread 47 extending along about half of its length towards its forward end, apart from a short forward portion 48. The forward portion 48 is unthreaded but is formed with several longitudinally-extending fins 49 the purpose of which is to prevent rotation of the column 44 by engagement with corresponding formations 50 in the casing while enabling the column to slide freely along its length. A cylindrical valve platform 51 having a screw thread on its inner surface (not shown) is screwed onto the column 44. The rear end of the platform 51 has an enlarged outwardly-extending flange 52, which is engaged by the forward end of a helical spring 53. The spring 53 embraces the column 44 and is contained within the housing 40 with its rear end engaging a ledge (not shown) within the housing. The spring 53, therefore, urges the column 44 forwardly relative to the housing 40 and urges the O-ring seal 46 into sealing engagement with the valve seat 43, which limits the forward extension of the column. It can be seen that, by adjusting the position of the valve platform 51 along the column 44 the force applied by the spring 53 can be increased or decreased.

The inspiratory valve 4 is adjustable between an opening pressure difference of 5 cmH₂O and 110 cmH₂O in increments of 1 cmH₂O. The valve pressure is set by the clinician and can be adjusted by sliding the lower moulding 25 forwardly so that the valve 4 is accessible. The clinician then rotates the valve housing 40 so as to rotate the valve platform 51 and move it longitudinally along the screw thread 47 on the valve column 44, thereby altering the compression of the spring 53. It would be preferable for the valve setting to be adjustable without having to open the apparatus so as to simplify setting and avoid exposing the clinician to any contamination within the apparatus. This could be achieved by a simple gear mechanism within the apparatus accessible by a key.

With reference to FIG. 2, the electronics housing 11 contains electronics 60 and provides a display representation to the user. The flat outer, lower surface 26′ of the housing 11 includes a transparent viewing screen 61 with two seven-segment LCD 62 display regions, or some other numerical display on which a count of the number of breaths taken within a defined period is represented. The main part of the housing 11 is of a translucent plastics and within this part is housed one or more lamps 63 (typically five LEDs) connected to the electronics 60. The mode of operation of the lamps 63 and numerical display 62 will be described later. The electronics unit 60 also includes some form of sensor 64 located adjacent the inspiratory valve assembly 4 to detect movement of the valve column 44. Preferably the valve column 44 incorporates a magnet and the sensor 64 in the electronics unit 60 is a Hall effect, reed switch or other form of magnet sensor. This arrangement enables the electronics housing 11 to be completely sealed. Alternative sensors could include an inductive coil to sense the presence of a ferromagnetic material, an optical sensor or the like.

The user picks up the apparatus by gripping the handle portion 10 and lifting the mouthpiece 1 up to his mouth with the viewing screen 61 lowermost. The user then places his mouth over the mouthpiece 1 and inhales. This creates a reduced pressure to the rear of the inspiratory valve 4. If the inspiratory breath was sufficiently forceful the pressure drop would be sufficient to overcome the force of the spring 53 and move the valve column 44 rearwardly a short distance, thereby lifting the seal 46 off the valve seat 43 and allowing air to flow through the valve 4 from the inlet 2. It will be appreciated that air is prevented from entering the apparatus via the outlet 3 by the one-way expiratory valve 34 in the saliva trap 27. When the user exhales, the inspiratory valve 4 closes so that the expired air flows down, opening the expiratory one-way valve 34, and out through the air outlet 3 via the saliva trap 27.

The movement of the valve column 44 is sensed by the electronics unit 60, which causes the apparatus to turn on and start a sequence of energising the LEDs 63 simultaneously to pulse on and off for the duration of the prescribed therapy period, typically of ten minutes. The LEDs 63 illuminate the interior of the electronics housing 11 and, because of its translucent nature, this causes the entire housing to pulse on and off in a manner that is visible to the user while he is exercising. The frequency of the pulsing is chosen to replicate the desired frequency of the inspiratory exercise, typically the lamps 63 glowing on for three seconds, during the inspiratory period, and then turning off for five seconds, during the expiratory period. The visual cue provided by this pulsing illumination is readily apparent to elderly users even if they are partially sighted and the pulsing illumination effect helps to steady the user's breathing rate. Audible signals, by contrast, could be more difficult especially for those who are hard of hearing. Ambient noise and the air flow noise produced by the apparatus during use could also make audible signals difficult to hear. At the end of the desired therapy period the electronics unit 60 is programmed to change the illumination effect to a steady state to indicate that the session has finished. Preferably the lamps 63 are energised to glow constantly with a mid level of illumination for a set period, typically five minutes, during which time the user can check his breath count. The user does this by turning the apparatus so that he can see the display on the screen clearly. The angle of the viewing screen 61 is chosen so that the numerical display 62 cannot be read during the therapy session but only after the user has completed his breathing exercises, removed the apparatus from his mouth and turned it over. The electronics unit 60 maintains a count of the number of breaths taken during the defined therapy period and increments the display 62 by one for each breathing cycle. The display 62 maintains the final count visible for the set period and then the apparatus powers off. Showing the breath count in this way encourages the user and builds his confidence as they see his breath count increase.

The electronics unit 60 maintains a record of operation of the apparatus, which can be downloaded to the clinician's computer by connection to a USB port 65 on the electronics housing 11. Alternatively, data could be transferred by wireless means, such as via an infra-red port or radio transmission.

The apparatus can be cleaned in various different ways, such as by autoclaving or in a domestic dishwasher. The electronics unit 60 is removed before cleaning and the inspiratory valve 4 is also removed before autoclaving. The saliva trap 27 should be removed and cleaned after each use.

The construction of the apparatus makes it particularly suitable for use with patients suffering from respiratory problems although the apparatus could also be used by athletes, divers, mountaineers, singers and wind musicians wanting to increase their inspiratory lung function. The display arrangement encourages regular use for the correct duration so as to ensure the maximum beneficial effect.

1-15. (canceled)

16. Respiratory therapy apparatus including a mouthpiece, an air opening and a restriction to flow of air between the mouthpiece and the opening against which the patient breathes, characterized in that the apparatus including means to record use of the apparatus and provide an indication thereof.

17. Apparatus according to claim 16, characterized in that the apparatus includes means by which the record of use of the apparatus can be downloaded to a remote computer.

18. Apparatus according to claim 17, characterized in that the means by which the record of use can be downloaded is selected form one or more of the following: an electrical connector port, an infra-red transmitter and a radio transmitter.

19. Apparatus according to claim 16, characterized in that the air opening is an inlet such that the patient can inhale air from the opening via the restriction.

20. Apparatus according to claim 16, characterized in that the restriction includes a valve with a spring urging a valve member against a valve seat.

21. Apparatus according to claim 20, characterized in that the force with which the valve member is urged against the valve seat is adjustable by rotating a part of the valve.

22. Apparatus according to claim 16, characterized in that the apparatus is arranged to indicate the duration of the prescribed therapy period.

23. Apparatus according to claim 22, characterized in that the apparatus is arranged to indicate the duration of the prescribed therapy period by visual means that changes between an alternating and a steady state.

24. Apparatus according to claim 23, characterized in that the visual means is arranged to alternate during the prescribed therapy period and changes to a steady state when the prescribed therapy period has ended.

25. Apparatus according to claim 23, characterized in that the visual means includes a lamp located within a translucent part of the housing of the apparatus so that the translucent part is illuminated by the lamp.

26. Apparatus according to claim 23, characterized in that the steady state is an illuminated state of a predetermine duration.

27. Apparatus according to claim 16, characterized in that the apparatus includes a display for representing a count of the number of breaths taken during the therapy period.

28. Apparatus according to claim 27, characterized in that the display of count is positioned such that it is not visible by the patient during the therapy period.

29. Apparatus according to claim 16, characterized in that the apparatus is of a generally looped shape including a first arm containing the air opening and the restriction to flow of air, a second arm including a second air opening and the means to record use of the apparatus, that the second arm is attached at one end with the first arm, extends alongside the first arm and is spaced from it to provide an open loop by which the apparatus can be supported, and that the mouthpiece extends generally in a direction opposite from the loop.

30. Apparatus according to claim 29, characterized in that the first and second arms are arranged such that the apparatus can be supported with both arms resting on a support surface and with the mouthpiece spaced above the surface.
